SEQUENCE LISTING (22 sequences)

SEQ ID NO: 1 - DNA, 249 bp, Homo sapiens
gaagatagaaggcgtgtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgggaagaagatccacatgaaggaaaaatatggttccatgggaagatttccaaacaggaagct

SEQ ID NO: 2 - DNA, 204 bp, Homo sapiens
gtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgggaagaagatccacatgaaggaaaaatatgg

SEQ ID NO: 3 - DNA, 174 bp, Homo sapiens
gtacgagctattctaccttacacaaaagtaccagacactgatgaaataagtttcttaaaaggagatatgttcattgttcataatgaattagaagatggatggatgtgggttacaaatttaagaacagatgaacaaggccttattgttgaagacctagtagaagaggtgggccgg

SEQ ID NO: 4 - DNA, 30 bp, Homo sapiens
tggatgtgggttacaaatttaagaacagat

SEQ ID NO: 5 - protein, 10 aa, Homo sapiens
WMWVTNLRTD

SEQ ID NO: 6 - protein, 10 aa, Bos taurus
WMWVTNLRTD

SEQ ID NO: 7 - protein, 10 aa, Mus musculus
WMWVTNLRTD

SEQ ID NO: 8 - protein, 10 aa, Rattus norvegicus
WMWVTNLRTD

SEQ ID NO: 9 - protein, 10 aa, Anopheles sp.
WLWVTAHRTG

SEQ ID NO: 10 - protein, 10 aa, Drosophila sp.
WLWVTAHRTG

SEQ ID NO: 11 - protein, 10 aa, Artificial
  Synthetic peptide
WLWVSNLRTD

SEQ ID NO: 12 - protein, 10 aa, Artificial
  synthetic peptide
WMWVTNHRTD

SEQ ID NO: 13 - protein, 10 aa, Artificial
  synthetic peptide
WXWVTXXRTX

SEQ ID NO: 14 - protein, 83 aa, Homo sapiens
EDRRRVRAILPYTKVPDTDEISFLKGDMFIVHNELEDGWMWVTNLRTDEQGLIVEDLVEEVGREEDPHEGKIWFHGKISKQEA

SEQ ID NO: 15 - protein, 69 aa, Homo sapiens
RVRAILPYTKVPDTDEISFLKGDMFIVHNELEDGWMWVTNLRTDEQGLIVEDLVEEVGREEDPHEGKIW

SEQ ID NO: 16 - protein, 59 aa, Homo sapiens
RVRAILPYTKVPDTDEISFLKGDMFIVHNELEDGWMWVTNLRTDEQGLIVEDLVEEVGR

SEQ ID NO: 17 - protein, 22 aa, Artificial
  synthetic peptide
GRKKRRQRRRGGWMWVTNLRTD

SEQ ID NO: 18 - DNA, 80 bp, Artificial
  synthetic sequence
aattcgccccatgggctacccgtacgacgtgccggactacgcttcttggatgtgggttacaaatttaagaacagattagg

SEQ ID NO: 19 - DNA, 80 bp, Artificial
  synthetic sequence
gatccctaatctgttcttaaatttgtaacccacatccaagaagcgtagtccggcacgtcgtacgggtagcccatggggcg

SEQ ID NO: 20 - protein, 10 aa, Artificial
  synthetic peptide
GRKKRRQRRR

SEQ ID NO: 21 - protein, 21 aa, Artificial
  synthetic peptide
RRRRRRRRRGGWMWVTNLRTD

SEQ ID NO: 22 - protein, 9 aa, Artificial
  synthetic sequence
RRRRRRRRR

(19) United States
(12) Patent Application Publication
(10) Pub. No.: US 2012/0022000 A1
(43) Pub. Date: Jan. 26, 2012

(54) USE OF A PEPTIDE IN THE TREATMENT OR PREVENTION OF METASTASIS

(75) Inventor: Christian Widmann, Lausanne (CH)

(73) Assignee: UNIVERSITE DE LAUSANNE, Lausanne (CH)

(21) Appl. No.: 13/147,296

(22) PCT Filed: Jan. 29, 2010
(86) PCT No.: PCT/IB2010/050403

§ 371 Date: Aug. 1, 2011

Related U.S. Application Data
(60) Provisional application No. 61/202,131, filed on Jan. 30, 2009.

(51) Int. Cl. A61K 38/08; C07K 7/06; C07K 14/47; A61P 35/00; A61K 38/02; C12N 5/071; A61P 35/04; C07K 2/00; A61K 38/17

(52) U.S. Cl. 514/19.8; 530/300; 530/328; 530/324; 514/1.1; 435/375

(57) ABSTRACT

The present invention relates to a peptide useful for the preparation of a medicament for the treatment or prevention of metastasis. Furthermore, it relates to a method of treatment or prevention of metastasis comprising administering to a subject in need thereof, a therapeutically effective amount of the peptide of the invention.

### FIELD OF THE INVENTION

The present invention relates to a peptide useful for the preparation of a medicament for the treatment or prevention of metastasis. Furthermore, it relates to a method of treatment or prevention of metastasis comprising administering to a subject in need thereof, a therapeutically effective amount of the peptide of the invention.

### BACKGROUND OF THE INVENTION

The dissemination of cancer cells away from the primary tumor is probably the event most dreaded by oncologists because the formation of metastases is the main cause of death from cancer. Metastasis occurs in various intricate stages (Fidler, 2003) that are now beginning to be understood at the molecular level (Zoller™, 2009; Weigelt and Peterse, 2005; Bogenrieder and Herlyn, 2003). Cells from a primary tumor need to achieve a series of tasks to eventually be able to promote metastatic colonization. They have to leave the tissue in which they arose. This requires modulation of their adhesion to their substratum, a capacity to degrade the extracellular matrix and a concomitant ability to migrate away. Once on the move they have to escape immune surveillance and withstand new environments favoring their death. They finally need to home to specific organs in which they promote lymphogenesis or angiogenesis to sustain their growth as metastases. Any impairment during these various stages can compromise the development of metastases. However, from a therapeutical point of view, it would appear more suited to inhibit the initial steps, namely modulation of cell adhesion, extracellular matrix degradation, and cell migration, to better control further potential development of the tumor, hence favoring patient survival i.e. if tumor cells cannot move away from the site of their appearance, relapses would be more easily identified and treated.

### SUMMARY OF THE INVENTION

This object has been achieved by providing the use of a peptide consisting essentially of the N2 sequence of the RasGAP protein, a biologically active fragment thereof, or a variant thereof, for the preparation of a medicament for the treatment or prevention of metastasis.

Furthermore, the invention provides a method of treatment or prevention of metastasis comprising administering to a subject in need thereof, a therapeutically effective amount of

i) a peptide consisting essentially of the N2 sequence of the RasGAP protein, a biologically active fragment thereof, or a variant thereof, or
ii) a peptide consisting essentially of the N2 sequence of the RasGAP protein, a biologically active fragment thereof, or a variant thereof, to a subject in need thereof, conjugated to an agent which increases the accumulation of said peptide in a cell.

The invention further provides an in vivo method of modulating the cell adhesion and cell migration comprising contacting a cell with the peptide of the invention, a biologically active fragment thereof or a variant thereof.

Also provided is a kit for treating or preventing metastasis in a subject, a kit for enhancing the cellular adhesion in vitro as well as the use of a peptide of the invention, a biologically active fragment thereof, or a variant thereof, for modulating the cell adhesion in vitro.

### DESCRIPTION OF THE FIGURES

FIG. 1: TAT-RasGAP₃₁₇₋₃₂₆ induces cell adhesion.

(a) U2OS cells were cultured until confluency and treated during 0 h, 1 h, 3 h, 8 h and 14 h with 13 μM TAT, 13 μM TAT-RasGAP₃₁₇₋₃₂₆ or without treatment. The cells were then trypsinized during 5 minutes (or not trypsinized), washed with PBS and stained with GIEMSA. Four pictures were taken per plate using a Zeiss Axioplan 2 microscope equipped with a 10× objective and the number of cells per surface area was determined. Representative images are shown. The graph represents the number of cells per mm² (mean±95% CI of 3 independent experiments). Scale bar=100 μm. (b) Trypsinization assay on cancerous cell lines (U2OS, HeLa, 4T1, HCT116, SAOS) and non-cancerous cell lines (HEK293T and HaCaT). Cells were treated and analyzed as in panel (a). Values for control and TAT treatments are shown literally as they were too low to be seen on the graphs. (c) Reversibility of the TAT-RasGAP₃₁₇₋₃₂₆ induced adhesion. Confluent U2OS cells were treated during 24 h with 13 μM TAT, 13 μM TAT-RasGAP₃₁₇₋₃₂₆ or not treated. The cells were then washed and incubated in culture medium without peptides for the indicated periods of time. Cells were analyzed as in panel (a).

FIG. 2: TAT-RasGAP₃₁₇₋₃₂₆ suppresses cell migration.

Cells were cultured until confluency and a wound in the cell layer was done with a yellow tip. The cells were then left untreated or incubated 24 hours with 13 μM of TAT or TAT-RasGAP₃₁₇₋₃₂₆ peptides. Pictures were taken just after wounding (0 hour) and at the indicated times. Results correspond to the width of the wound (mean±95% CI of 4 independent experiments). Asterisks denote significant differences between the control and the other conditions at a given time point (t-test after Bonferroni correction). Bar=100 μm.

FIG. 3: TAT-RasGAP₃₁₇₋₃₂₆ does not affect cell culture growth.

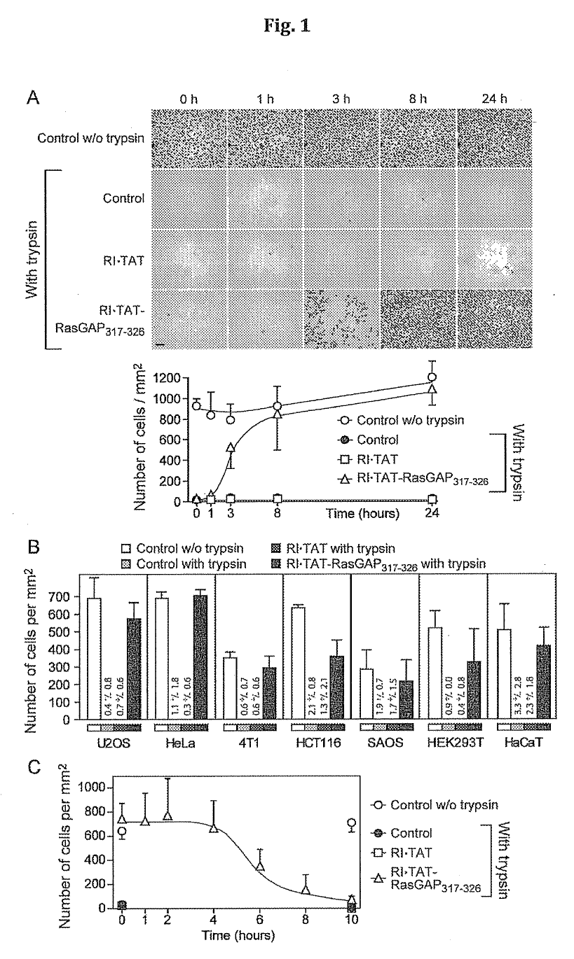

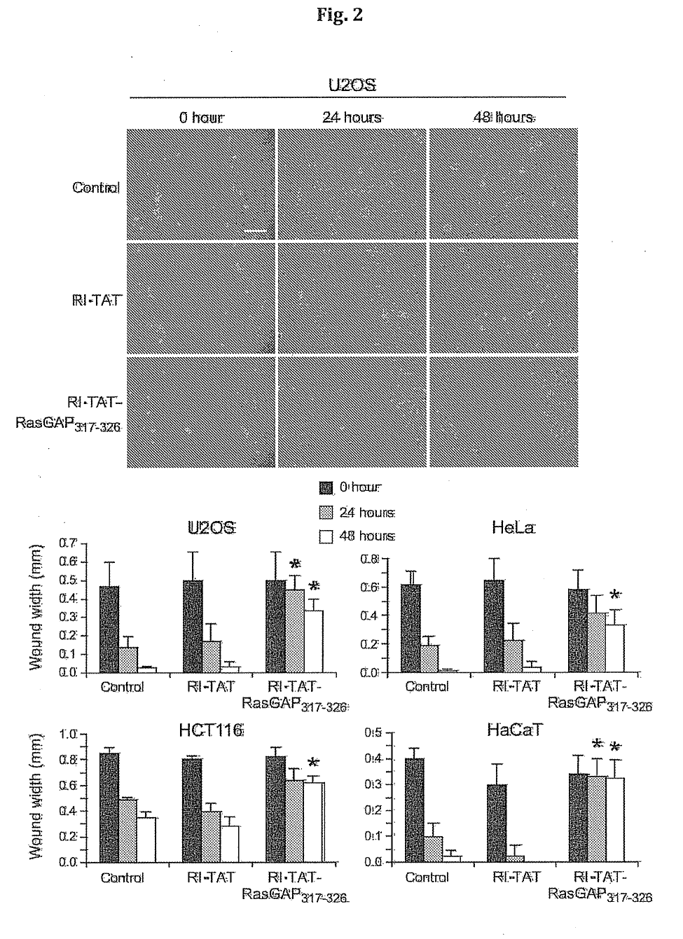

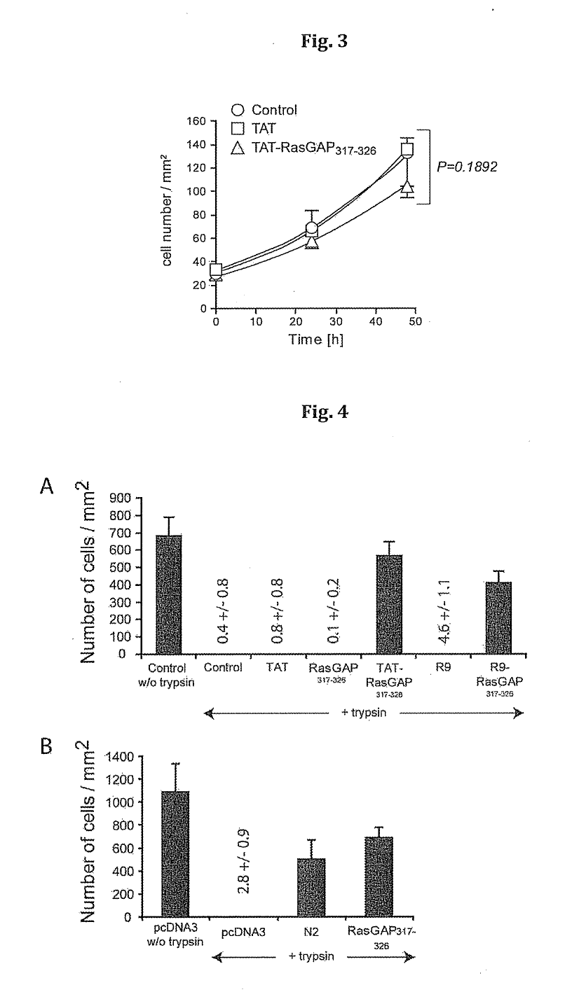

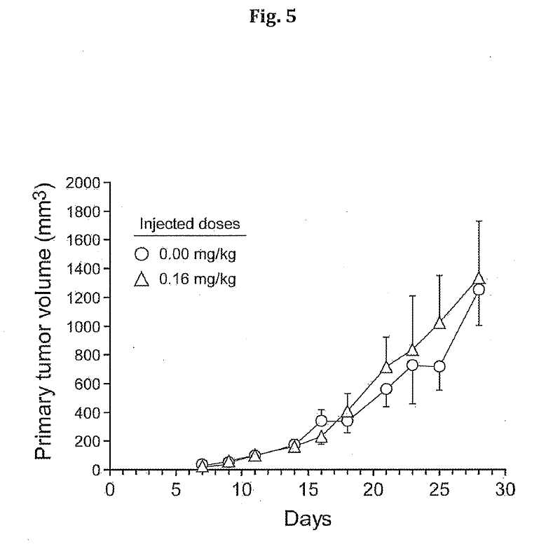

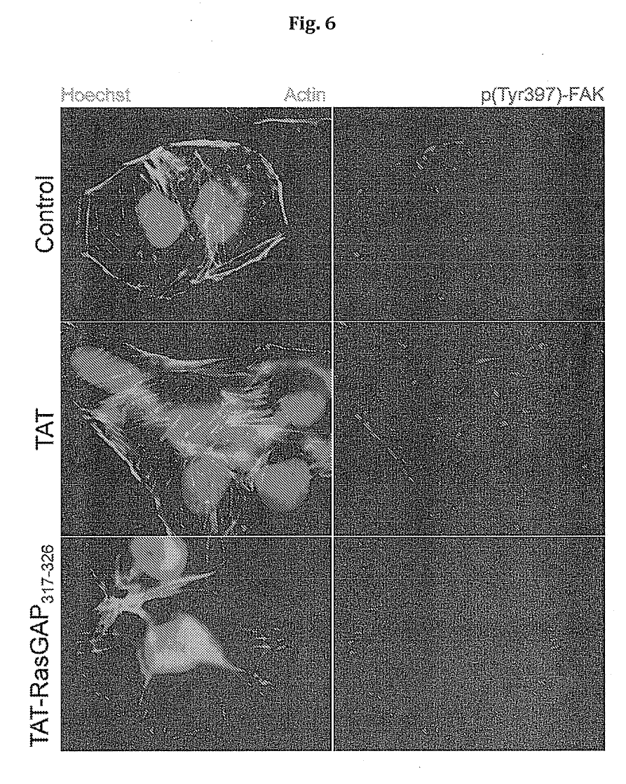

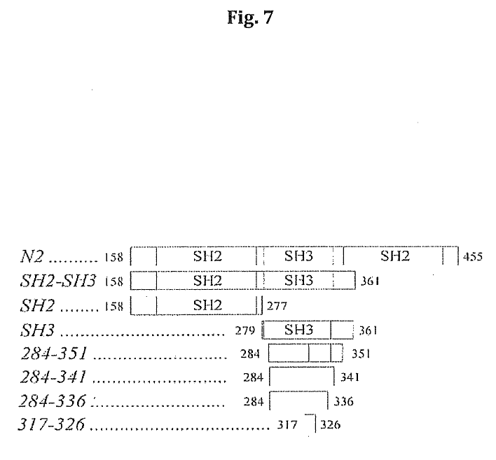

30,000 U2OS cells were seeded in 3.5 cm-plates. The cell number in the plates were then determined 24 and 48 hours later as described in FIG. 1, panel (a). The graph represents the number of cells per mm2 (mean±95% CI of 3 independent experiments). No significant difference between the growth curves could be detected (repeated measures ANOVA).

FIG. 4: RasGAP₃₁₇₋₃₂₆ acts from inside the cell.

(a) RasGAP317-326 without cell-permeation sequences is not able to render cells resistant to trypsin. 300,000 U2OS cells were cultured overnight in 3.5 cm plates and then treated during 8 hours with the indicated peptides (the concentration of each peptide was 13 μM). The cells were then subjected to a trypsinization assay. (b) 2×106 HEK293T cells were cultured overnight in 10 cm plates and transfected with empty vector (pcDNA3) or vectors encoding HA-tagged versions of fragment N2 or the 317-326 sequence of RasGAP. One day later, the cells were subjected to a trypsinization test. Results in (a) and (b) correspond to the number of cells per mm2 (mean±95% CI of 3 independent experiments). Some values are shown literally as they were too low to be seen on the graphs.

FIG. 5: TAT-RasGAP₃₁₇₋₃₂₆ does not affect primary tumor growth in an orthotopic fatpad tumor model.

100,000 4T1-luc2 cells were injected orthotopically into the mammary fatpad of nude mice. Four groups of eight nude mice were then treated with or without 0.16 mg TAT-RasGAP317-326 per kg of mouse on the first, third and fifth day of the week during 28 days. The tumor volumes were measured with a caliper at the indicated time points and were calculated according to the formula described in the “MATERIAL AND METHODS” section. A repeated measure ANOVA was performed and no differences were found between the two groups.

FIG. 6: Actin and focal adhesion changes in TAT-RasGAP₃₁₇₋₃₂₆-treated cells. 100,000 U2OS cells were cultured on coverslips for 24 hours and then treated with 13 μM TAT-RasGAP₃₁₇₋₃₂₆, 13 μM TAT or left untreated (control) over-night. The cells were then fixed with 2% PFA, and peinieabilized with PBS Triton-X100 0.2%. The nuclei were stained with Hoechst 33342 and actin with Alexa Fluor 488 phalloidin. Focal adhesions were stained with an anti-phospho Tyr397 FAK. Representative images are shown.

FIG. 7: Schematic representation of the different constructs used in this study.

SH represents the Src homology domain.

### DETAILED DESCRIPTION OF THE INVENTION

The present invention relates to the use of a peptide consisting essentially of the N2 sequence of the RasGAP protein, a biologically active fragment thereof, or a variant thereof, for the preparation of a medicament for the treatment or prevention of metastasis.

As used herein, the terms “peptide”, “protein”, “polypeptide”, “polypeptidic” and “peptidic” are used interchangeably to designate a series of amino acid residues connected to the other by peptide bonds between the alpha-amino and carboxy groups of adjacent residues.

“Metastasis” is the spread of a malignant tumor cells from one organ or part to another non-adjacent organ or part. Cancer cells can “break away”, “leak”, or “spill” from a primary tumor, enter lymphatic and blood vessels, circulate through the bloodstream, and settle down to grow within normal tissues elsewhere in the body. Metastasis is one of three hallmarks of malignancy (contrast benign tumors). Most tumors and other neoplasms can metastasize, although in varying degrees (e.g., glioma and basal cell carcinoma rarely metastasize). When tumor cells metastasize, the new tumor is called a secondary or metastatic tumor.

By “cancer cell” is meant a cell arising in an animal in vivo which is capable of undesired and unregulated cell growth or abnormal persistence or abnormal invasion of tissues. In vitro this term also refers to a cell line that is a permanently immortalized established cell culture that will proliferate indefinitely and in an unregulated manner given appropriate fresh medium and space.

RasGAP, a regulator of Ras and Rho GTP-binding proteins, is an unconventional caspase substrate because it can induce both anti- and pro-apoptotic signals, depending on the extent of its cleavage by caspases. At low levels of caspases, RasGAP is cleaved at position 455, generating an N-terminal fragment (fragment N, of about 56 kD) and a C-terminal fragment (fragment C, of about 64 kD). Fragment N appears to be a general blocker of apoptosis downstream of caspase activation (Yang J.-Y. and Widmann C., Mol. Cell. Biol., 21, 5346, 2001 and J. Biol. Chem., 277, 14641, 2002b). At high levels of caspase activity, fragment N is further cleaved at position 157 thus generating two fragments, N1 (amino acids 1 to 157) and N2 (amino acids 158 to 455).

The N2 sequence of the RasGAP protein, when derived from human, refers to a 36 kD protein consisting of 297 amino acids which encompasses two SH2 and one SH3 domain as shown in FIG. 7. In general, Src homology 2 (SH2) domains are involved in recognition of phosphorylated tyrosine whereas Src homology 3 (SH3) domains are often indicative of a protein involved in signal transduction.

“A biologically active fragment of the N2 sequence of the RasGAP protein” refers to a sequence containing less amino acids in length than the N2 sequence of the RasGAP protein. This sequence can be used as long as it exhibits the same biological properties as the native sequence from which it derives. Preferably this sequence contains less than 90%, preferably less than 60%, in particular less than 30% amino acids in length than the respective N2 sequence of the RasGAP protein.

The present invention also includes the use of a variant of the N2 sequence of the RasGAP protein as well as of the biologically active fragment of the N2 sequence. The term “variant” refers to a peptide having an amino acid sequence that differ to some extent from a native sequence peptide, that is an amino acid sequence that vary from the native sequence by conservative amino acid substitutions, whereby one or more amino acids are substituted by another with same characteristics and conformational roles. The amino acid sequence variants possess substitutions, deletions, and/or insertions at certain positions within the amino acid sequence of the native amino acid sequence. Conservative amino acid substitutions are herein defined as exchanges within one of the following five groups:

I. Small aliphatic, nonpolar or slightly polar residues: Ala, Ser, Thr, Pro, Gly
II. Polar, positively charged residues: H is, Arg, Lys
III. Polar, negatively charged residues: and their amides: Asp, Asn, Glu, Gln
IV. Large, aromatic residues: Phe, Tyr, Trp
V. Large, aliphatic, nonpolar residues: Met, Leu, Ile, Val, Cys.

The N2 sequence, as well as a fragment and a variant thereof can be prepared by a variety of methods and techniques known in the art such as for example chemical synthesis or recombinant techniques as described in Maniatis et al. 1982, Molecular Cloning, A laboratory Manual, Cold Spring Harbor Laboratory.

Preferably, the biologically active fragment of the N2 sequence of the RasGAP protein comprises the amino acid sequence of the SH3 domain of the N2 sequence, a part thereof, or a variant thereof.

Applicants have characterized shorter sequences of the N2 sequence of the RasGAP protein that, surprisingly, still block the migration capacity of tumor cells by increasing to a great extent the adherence capacity of tumor cells and their ability to migrate (see Example 1).

They have then generated progressive truncations in the SH3 domain in an attempt to identify a minimal biologically active sequence. All these constructs or parts of the N2 sequence (FIG. 7), including the shortest one (317-326) that codes for a 10 amino acid long peptide, that still block the migration capacity of cells, in particular tumor or cancer cells. These results show that the biological property of fragment N2 does not require a complete SH3 domain but is mediated by a part of the SH3 domain such as a short peptidic sequence.

Thus the biologically active fragment of the SH3 domain or the variant thereof contains preferably less than or equal to 70, more preferably less than or equal to 30, most preferably less than or equal to 10 amino acids of the amino acid sequence of the SH3 domain.

In particular, encompassed by the present invention, is a biologically active fragment of the SH3 domain which consists in an amino acid sequence encoded by a DNA sequence selected from the sequences of Table 1:

TABLE 1

| DNA | | | Amino acid |
|---|---|---|---|
| Sequence | | | sequences |
| ID | Name | DNA sequences | (SEQ ID N^{o}) |
| SEQ ID N^{o} 1 | RasGAP₂₈₄₋₃₅₁ | gaagatagaaggcgtgtacgagctattctacctta | EDRRRVRAILPYTKV |
| | | cacaaaagtaccagacactgatgaaataagtttct | PDTDEISFLKGDMFI |
| | | taaaaggagatatgttcattgttcataatgaatta | VHNELEDGWMWVTNL |
| | | gaagatggatggatgtgggttacaaatttaagaac | RTDEQGLIVEDLVEE |
| | | agatgaacaaggccttattgttgaagacctagtag | VGREEDPHEGKIWFH |
| | | aagaggtgggccgggaagaagatccacatgaagga | GKISKQEA |
| | | aaaatatggttccatgggaagatttccaaacagga | (SEQ ID N^{o} 14) |
| | | agct | |
| SEQ ID N^{o} 2 | RasGAP₂₈₄₋₃₄₁ | gtacgagctattctaccttacacaaaagtaccaga | RVRAILPYTKVPDTD |
| | | cactgatgaaataagtttcttaaaaggagatatgt | EISFLKGDMFIVHNE |
| | | tcattgttcataatgaattagaagatggatggatg | LEDGWMWVTNLRTDE |
| | | tgggttacaaatttaagaacagatgaacaaggcct | QGLIVEDLVEEVGRE |
| | | tattgttgaagacctagtagaagaggtgggccggg | EDPHEGKIW |
| | | aagaagatccacatgaaggaaaaatatgg | (SEQ ID N^{o} 15) |
| SEQ ID N^{o} 3 | RasGAP₂₈₄₋₃₃₆ | gtacgagctattctaccttacacaaaagtaccaga | RVRAILPYTKVPDTD |
| | | cactgatgaaataagtttcttaaaaggagatatgt | EISFLKGDMFIVHNE |
| | | tcattgttcataatgaattagaagatggatggatg | LEDGWMWVTNLRTDE |
| | | tgggttacaaatttaagaacagatgaacaaggcct | QGLIVEDLVEEVGR |
| | | tattgttgaagacctagtagaagaggtgggccgg | (SEQ ID N^{o}16) |
| SEQ ID N^{o} 4 | RasGAP₃₁₇₋₃₂₆ | tggatgtgggttacaaatttaagaacagat | WMWVTNLRTD |
| | | | (SEQ ID N^{o} 5) |

In case the part of the SH3 domain of the N2 sequence is SEQ ID No. 4 (RasGAP₃₁₇₋₃₂₆) then the resulting amino acid sequence encoded by said SEQ ID No. 4 in human is WMWVTNLRTD.

A comparison between the different species revealed that there are different amino acids, which are conserved among the species as shown in table 2.

TABLE 2

| | Amino acid | |
|---|---|---|
| | sequences of | Amino acid |
| Species | RasGAP₃₁₇₋₃₂₆ | Sequence ID |
| Human | WMWVTNLRTD | SEQ ID N^{o} 5 |
| Bos taurus | WMWVTNLRTD | SEQ ID N^{o} 6 |
| Mouse | WMWVTNLRTD | SEQ ID N^{o} 7 |
| Rattus norvegicus | WMWVTNLRTD | SEQ ID N^{o} 8 |
| Anopheles | WLWVTAHRTG | SEQ ID N^{o} 9 |
| Drosophilia | WLWVTAHRTG | SEQ ID N^{o} 10 |
| Variant 1* | WLWVSNLRTD | SEQ ID N^{o} 11 |
| Variant 2* | WMWVTNHRTD | SEQ ID N^{o} 12 |
| Alignment | WxWVTxxRTx | SEQ ID N^{o} 13 |

*Variants 1 and 2 are synthetic peptides and are not found in biological species

Conserved amino acids among the species are represented as bold underlined type residues whereas the X correspond to amino acid residues that can be changed by conservative, or non-conservative amino acid substitutions, without impairing the inventive and biological properties of these 10 amino acid parts of the SH3 domain of N2.

These peptidic variants of this 10 amino acid part of the human SH3 domain of N2, and in particular the alignment sequence WXWVTXXRIRX, are also encompassed by the present invention and they refer to peptides having an amino acid sequence that differ to some extent from the native sequence peptide, that is the amino acid sequence that vary from the native sequence WMWVTNLRTD by conservative or non-conservative amino acid substitutions, whereby one or more amino acid residues are substituted by another with same characteristics and conformational roles.

Preferably, the biologically active fragment comprising the amino acid sequence of the SH3 domain of the N2 sequence comprises the general amino acid sequence WXWVTXXRTX (SEQ ID No. 13), wherein X represents an amino acid.

Preferably also the biologically active fragment comprising the amino acid sequence of the SH3 domain of the N2 sequence consists in an amino acid sequence encoded by a DNA sequence selected from the group comprising SEQ ID No. 1, SEQ ID No. 2, SEQ ID No. 3 or SEQ ID No. 4 or consists in an amino acid sequence selected from the groups comprising SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, or SEQ ID No. 15.

Usually, the peptide consisting essentially of the N2 sequence of the RasGAP protein, a biologically active fragment thereof, or a variant thereof as disclosed in the present invention is conjugated to an agent which increases the accumulation of the peptide in a cell.

Such an agent can be a compound which induces receptor mediated endocytose such as for example the membrane transferrin receptor mediated endocytosis of transferrin conjugated to therapeutic drugs (Qian et al., 2002) or a cell membrane permeable carrier which can, be selected e.g. among the group of fatty acids such as decanoic acid, myristic acid and stearic acid, which have already been used for intracellular delivery of peptide inhibitors of protein kinase C (Ioannides et al., 1990) and protein-tyrosine phosphatase (Kole et al., 1996) or among peptides. Preferably, cell membrane permeable carriers are used, more preferably a cell membrane permeable carrier peptide is used.

In case the cell membrane permeable carrier is a peptide then it will preferably be a positively charged amino acid rich peptide.

Preferably such positively charged amino acid rich peptide is an arginine rich peptide. It has been recently shown in Futaki et al. (Futaki S. et al., 2001), that the number of arginine residues in a cell membrane permeable carrier peptide has a significant influence on the method of internalization and that there seems to be an optimal number of arginine residues for the internalization. Accordingly, the positively charged amino acid rich peptide will preferably contain more than 6 arginines, more preferably it contains 7 arginines, even more preferably 8 arginines and even more preferably it contains 9 arginines.

The peptide of the invention may be conjugated to the cell membrane permeable carrier by a spacer. In this case the cell membrane permeable carrier is preferably a peptide.

Usually arginine rich peptides are selected from the group comprising the HIV-TAT₄₈₋₅₇ peptide, the FHV-coat₃₅₋₄₉ peptide, the HTLV-II Rex₄₋₁₆ peptide and the BMV gag₇₋₂₅ peptide. Preferably, the arginine rich peptide is either the HIV-TAT₄₈₋₅₇ peptide or the R9 peptide (SEQ ID No 17: RRRRRRRRR).

In case the HIV-TAT₄₈₋₅₇ peptide or the R9 peptide is conjugated to a RasGAP sequence, such as for example RasGAP₃₁₇₋₃₂₆, then two glycine residues are usually inserted between the TAT or the R9 and RasGAP sequences as spacer to allow flexibility.

Since an inherent problem with native peptides (in L-form) is degradation by natural proteases, the peptide of the invention may be prepared to include D-forms and/or “retro-inverso isomers” of the peptide.

In this case, retro-inverso isomers of fragments and variants of the peptide of the invention are prepared.

Protecting the peptide from natural proteolysis should therefore increase the effectiveness of the specific heterobivalent or heteromultivalent compound. A higher biological activity is predicted for the retro-inverso containing peptide when compared to the non-retro-inverso containing analog owing to protection from degradation by native proteinases. Furthermore they have been shown to exhibit an increased stability and lower immunogenicity (Sela and Zisman, 1997).

Retro-inverso peptides are prepared for peptides of known sequence as described for example in Sela and Zisman.

By “retro-inverso isomer” is meant an isomer of a linear peptide in which the direction of the sequence is reversed and the chirality of each amino acid residue is inverted; thus, there can be no end-group complementarity.

Also encompassed by the present invention are modifications of the peptide (which do not normally alter primary sequence), including in vivo or in vitro chemical derivitization of peptides, e.g., acetylation or carboxylation. Also included are modifications of glycosylation, e.g., those made by modifying the glycosylation patterns of a peptide during its synthesis and processing or in further processing steps, e.g., by exposing the peptide to enzymes which affect glycosylation e.g., mammalian glycosylating or deglycosylating enzymes. Also included are sequences which have phosphorylated amino acid residues, e.g., phosphotyrosine, phosphoserine, or phosphothreonine.

The invention also includes analogs in which one or more peptide bonds have been replaced with an alternative type of covalent bond (a “peptide mimetic”) which is not susceptible to cleavage by peptidases. Where proteolytic degradation of the peptides following injection into the subject is a problem, replacement of a particularly sensitive peptide bond with a noncleavable peptide mimetic will make the resulting peptide more stable and thus more useful as an active substance. Such mimetics, and methods of incorporating them into peptides, are well known in the art.

Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4,-dinitrophenyl. Blocking the charged amino- and carboxy-termini of the peptides would have the additional benefit of enhancing passage of the peptide through the hydrophobic cellular membrane and into the cell.

When recombinant techniques are employed to prepare a peptide consisting essentially of the N2 sequence of the RasGAP protein, a biologically active fragment thereof, or a variant thereof, in accordance with the present invention, nucleic acid sequences encoding the polypeptides are preferably used. With regard to the method to practise recombinant techniques, see for example, Maniatis et al. 1982, Molecular Cloning, A laboratory Manual, Cold Spring Harbor Laboratory and commercially available methods.

Accordingly the present invention also relates to a purified and isolated nucleic acid sequence encoding a peptide consisting essentially of the N2 sequence of the RasGAP protein, a biologically active fragment thereof, or a variant thereof as described above.

This purified and isolated nucleic acid sequence can be used in transfection methods. As can be shown from example 2, transfecting cells with a plasmid encoding the RasGAP₃₁₇₋₃₂₆ sequence also rendered cells more adherent (FIG. 4B), demonstrating that cells can increase their adherence if they synthesize their own RasGAP₃₁₇₋₃₂₆ peptide.

“A purified and isolated nucleic acid or nucleic acid sequence” refers to the state in which the nucleic acid sequence encoding the peptide of the invention, or nucleic acid encoding such peptide consisting essentially of the N2 sequence of the RasGAP protein, a biologically active fragment thereof, or a variant thereof will be, in accordance with the present invention. A purified and isolated nucleic acid or nucleic acid sequence encompassed by the present invention might be DNA, RNA, or DNA/RNA hybrid.

DNA which can be used herein is any polydeoxynucleotide sequence, including, e.g. double-stranded DNA, single-stranded DNA, double-stranded DNA wherein one or both strands are composed of two or more fragments, double-stranded DNA wherein one or both strands have an uninterrupted phosphodiester backbone, DNA containing one or more single-stranded portion(s) and one or more double-stranded portion(s), double-stranded DNA wherein the DNA strands are fully complementary, double-stranded DNA wherein the DNA strands are only partially complementary, circular DNA, covalently-closed DNA, linear DNA, covalently cross-linked DNA, cDNA, chemically-synthesized DNA, semi-synthetic DNA, biosynthetic DNA, naturally-isolated DNA, enzyme-digested DNA, sheared DNA, labeled DNA, such as radiolabeled DNA and fluorochrome-labeled DNA, DNA containing one or more non-naturally occurring species of nucleic acid.

DNA sequences that encode a peptide consisting essentially of the N2 sequence of the RasGAP protein, a biologically active fragment thereof, or a variant thereof, can be synthesized by standard chemical techniques, for example, the phosphodiester method or via automated synthesis methods and PCR methods.

The purified and isolated DNA sequence encoding a peptide consisting essentially of the N2 sequence of the RasGAP protein, a biologically active fragment thereof, or a variant thereof, according to the invention may also be produced by enzymatic techniques. Thus, restriction enzymes, which cleave nucleic acid molecules at predefined recognition sequences can be used to isolate nucleic acid sequences from larger nucleic acid molecules containing the nucleic acid sequence, such as DNA (or RNA) that codes for a peptide consisting essentially of the N2 sequence of the RasGAP protein, a biologically active fragment thereof, or a variant thereof.

Encompassed by the present invention is also a nucleic acid in the form of a polyribonucleotide (RNA), including, e.g., single-stranded RNA, cRNA, double-stranded RNA, double-stranded RNA wherein one or both strands are composed of two or more fragments, double-stranded RNA wherein one or both strands have an uninterrupted phosphodiester backbone, RNA containing one or more single-stranded portion(s) and one or more double-stranded portion(s), double-stranded RNA wherein the RNA strands are fully complementary, double-stranded RNA wherein the RNA strands are only partially complementary, covalently crosslinked RNA, enzyme-digested RNA, sheared RNA, mRNA, chemically-synthesized RNA, semi-synthetic RNA, biosynthetic RNA, naturally-isolated RNA, labeled RNA, such as radiolabeled RNA and fluorochrome-labeled RNA, RNA containing one or more non-naturally-occurring species of nucleic acid.

Preferably used as nucleic acid is a purified and isolated DNA sequence selected from the group comprising SEQ ID No. 1, SEQ ID No. 2, SEQ ID No. 3, or SEQ ID No. 4.

The present invention also includes variants of the aforementioned sequences, that is nucleotide sequences that vary from the reference sequence by conservative nucleotide substitutions, whereby one or more nucleotides are substituted by another with same characteristics.

The invention also encompasses allelic variants of the disclosed purified and isolated nucleic sequence; that is, naturally-occurring alternative forms of the isolated and purified nucleic acid that also encode peptides that are identical, homologous or related to that encoded by the purified and isolated nucleic sequences. Alternatively, non-naturally occurring variants may be produced by mutagenesis techniques or by direct synthesis.

The aforementioned purified and isolated nucleic acid sequence encoding a peptide consisting essentially of the N2 sequence of the RasGAP protein, a biologically active fragment thereof, or a variant thereof, may further comprise a nucleotide sequence encoding a cell membrane permeable carrier peptide.

Yet another concern of the present invention is to provide an expression vector comprising at least one copy of the isolated and purified nucleic acid sequence encoding a peptide consisting essentially of the N2 sequence of the RasGAP protein, a biologically active fragment thereof; or a variant thereof as described above. Preferably the isolated and purified nucleic acid sequence encoding a peptide of the invention is DNA.

As used herein, “vector”, “plasmid” and “expression vector” are used interchangeably, as the plasmid is the most commonly used vector form.

The vector may further comprise a nucleotide sequence encoding a cell membrane permeable carrier peptide in accordance with the invention. The choice of an expression vector depends directly, as it is well known in the art, on the desired functional properties, e.g., peptide expression and the host cell to be transformed or transfected.

Additionally, the expression vector may further comprise a promoter operably linked to the purified and isolated DNA sequence. This means that the linked isolated and purified DNA sequence encoding the peptide of the present invention is under control of a suitable regulatory sequence which allows expression, i.e. transcription and translation of the inserted isolated and purified DNA sequence.

As used herein, the term “promoter” designates any additional regulatory sequences as known in the art e.g. a promoter and/or an enhancer, polyadenylation sites and splice junctions usually employed for the expression of the polypeptide or may include additionally one or more separate targeting sequences and may optionally encode a selectable marker. Promoters which can be used provided that such promoters are compatible with the host cell are e.g promoters obtained from the genomes of viruses such as polyoma virus, adenovirus (such as Adenovirus 2), papilloma virus (such as bovine papilloma virus), avian sarcoma virus, cytomegalovirus (such as murine or human cytomegalovirus immediate early promoter), a retrovirus, hepatitis-B virus, and Simian Virus 40 (such as SV 40 early and late promoters) or promoters obtained from heterologous mammalian promoters, such as the actin promoter or an immunoglobulin promoter or heat shock promoters.

Enhancers which can be used are e.g. enhancer sequences known from mammalian genes (globin, elastase, albumin, a-fetoprotein, and insulin) or enhancer from a eukaryotic cell virus. e.g. the SV40 enhancer, the cytomegalovirus early promoter enhancer, the polyoma, and adenovirus enhancers.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pcDNA3, pMB9 and their derivatives, plasmids such as RP4; phage DNAs, e.g., the numerous derivatives of phage X, e.g., NM989, and other phage DNA, e.g., M13 and filamentous single stranded phage DNA; yeast plasmids such as the 2μ plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like. Most preferably the expression vector is pcDNA3.

Another concern of the present invention is to provide a eukaryotic or prokaryotic host cell containing the peptide according to the invention, the isolated and purified nucleic acid sequence of the invention or and/or expression vector described herein.

Transformation or transfection of appropriate eukaryotic or prokaryotic host cells with an expression vector comprising a purified and isolated DNA sequence according to the invention is accomplished by well known methods that typically depend on the type of vector used. With regard to these methods, see for example, Maniatis et al. 1982, Molecular Cloning, A laboratory Manual, Cold Spring Harbor Laboratory and commercially available methods. The term “cell transfected” or “cell transformed” or “transfected/transformed cell” means the cell into which the extracellular DNA has been introduced and thus harbours the extracellular DNA. The DNA might be introduced into the cell so that the nucleic acid is replicable either as a chromosomal integrant or as an extra chromosomal element.

The peptide consisting essentially of the N2 sequence of the RasGAP protein, a biologically active fragment thereof, or a variant thereof, optionally conjugated to an agent which increases the accumulation of the peptide in a cell as described herein are preferably produced, recombinantly, in a cell expression system. A wide variety of unicellular host cells are useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1. 1, B-W and L-M cells, African Green Monkey kidney cells (e.g., COS 1, COS 7, BSC1, BSC40, and BMT10), insect cells (e.g., Sf9), and human cells and plant cells in tissue culture. Preferably, the host cell is a bacterial cell, more preferably an E. coli cell.

Usually the medicament of the invention comprises a pharmaceutically effective amount of the peptide of the invention. “A pharmaceutically effective amount” refers to a chemical material or compound which, when administered to a human or animal organism induces a detectable pharmacologic and/or physiologic effect.

The respective pharmaceutically effect amount can depend on the specific patient to be treated, on the disease to be treated and on the method of administration. Further, the pharmaceutically effective amount depends on the specific peptide used. The treatment usually comprises a multiple administration of the pharmaceutical composition, usually in intervals of several hours, days or weeks. The pharmaceutically effective amount of a dosage unit of the peptide of the invention usually is in the range of 0.001 ng to 1000 μg per kg, preferably in the range of 0.001 ng to 100 μg per kg, of body weight of the patient to be treated.

Preferably, in addition to at least one peptide as described herein, the pharmaceutical composition may contain one or more pharmaceutically acceptable carriers, diluents and adjuvants. Acceptable carriers, diluents and adjuvants which facilitates processing of the active compounds into preparation which can be used pharmaceutically are non-toxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl orbenzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN®, PLURONICS® or polyethylene glycol (PEG).

The form of administration of the pharmaceutical composition may be systemic or topical. For example, administration of such a composition may be various parenteral routes such as subcutaneous, intravenous, intradermal, intramuscular, intraperitoneal, intranasal, transdermal, buccal routes or via an implanted device, and may also be delivered by peristaltic means.

The pharmaceutical composition comprising a peptide, as described herein, as an active agent may also be incorporated or impregnated into a bioabsorbable matrix, with the matrix being administered in the form of a suspension of matrix, a gel or a solid support. In addition the matrix may be comprised of a biopolymer.

Sustained-release preparations may be prepared. Suitable examples of sustained-release preparations include semi permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and [gamma]ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished for example by filtration through sterile filtration membranes.

It is understood that the suitable dosage of a peptide of the present invention will be dependent upon the age, sex, health, and weight of the recipient, kind of concurrent treatment, if any and the nature of the effect desired.

The appropriate dosage form will depend on the disease, the kind and origin of metastasis, the peptide, and the mode of administration; possibilities include tablets, capsules, lozenges, dental pastes, suppositories, inhalants, solutions, ointments and parenteral depots.

Since amino acid modifications of the amino acids of the peptide are also encompassed in the present invention, this may be useful for cross-linking the peptide of the invention to a water-insoluble matrix or the other macromolecular carriers, or to improve the solubility, adsorption, and permeability across the blood brain barrier. Such modifications are well known in the art and may alternatively eliminate or attenuate any possible undesirable side effect of the peptide and the like.

While a preferred pharmaceutical composition of the present invention comprises a peptide as an active agent, an alternative pharmaceutical composition may contain a purified and isolated nucleic acid sequence encoding the peptide, as described herein, as an active agent. This pharmaceutical composition may include either the sole purified and isolated DNA sequence, an expression vector comprising said purified and isolated DNA sequence or a host cell previously transfected or transformed with an expression vector described herein. In this latter example, host cell will preferably be isolated from the patient to be treated in order to avoid any antigenicity problem. These gene and cell therapy approaches are especially well suited for patients requiring repeated administration of the pharmaceutical composition, since the said purified and isolated DNA sequence, expression vector or host cell previously transfected or transformed with an expression vector can be incorporated into the patient's cell which will then produce the protein endogenously.

The peptide consisting essentially of the N2 sequence of the RasGAP protein, a biologically active fragment thereof, or a variant thereof, will generally be used in an amount to achieve the intended purpose. For use to treat or prevent metastasis, the peptide or a pharmaceutical composition thereof or a medicament, is administered or applied in a therapeutically effective amount. A “therapeutically effective amount” is an amount effective to ameliorate or prevent the symptoms, or prolong the survival of the subject being treated. Determination of a therapeutically effective amount is well within the capabilities of those skilled in the art, especially in light of the detailed disclosure provided herein.

“Administering”, as it applies in the present invention, refers to contact of the pharmaceutical composition, usually in the form of a therapeutically or pharmaceutically effective amount, to the subject, preferably a human.

For systemic administration, a therapeutically effective amount or dose can be estimated initially from in vitro assays. For example, a dose can be formulated in animal models to achieve a circulating concentration range that includes the IC50 as determined in cell culture. Such information can be used to more accurately determine useful doses in humans.

Initial doses can also be estimated from in vivo data, e.g. animal models, using techniques that are well known in the art. One ordinarily skill in the art could readily optimise administration to humans based on animal data and will, of course, depend on the subject being treated, on the subject's weight, the severity of the disorder, the manner of administration and the judgement of the prescribing physician.

The present disclosure also provides a method of treatment or prevention of metastasis comprising administering to a subject in need thereof, a therapeutically effective amount of

i) a peptide consisting essentially of the N2 sequence of the RasGAP protein, a biologically active fragment thereof, or a variant thereof, or
ii) a peptide consisting essentially of the N2 sequence of the RasGAP protein, a biologically active fragment thereof, or a variant thereof, to a subject in need thereof, conjugated to an agent which increases the accumulation of said peptide in a cell.

Examples of metastasis are those deriving from cancers such as carcinoma, lymphoma, blastoma, sarcoma, liposarcoma, neuroendocrine tumor, mesothelioma, schwanoma, meningioma, adenocarcinoma, melanoma, leukemia, lymphoid malignancy, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, testicular cancer, esophageal cancer, a tumor of the biliary tract, and head and neck cancer.

In preferred methods, the subject is a human patient, and the administered peptide is selected from the group comprising TAT-RasGAP₃₁₇₋₃₂₆ peptide and R9—RasGAP₃₁₇₋₃₂₆ peptide. The therapeutically effective amount of a dosage unit of the peptide of the invention is usually in the range of 0.001 ng to 1000 μg per kg, preferably in the range of 0.001 ng to 100 μg per kg of body weight of the human patient to be treated.

Embraced by the scope of the present invention is also an in vivo method of modulating the cell adhesion and cell migration comprising contacting a cell with at least one peptide consisting essentially of the N2 sequence of the RasGAP protein, a biologically active fragment thereof, or a variant thereof, conjugated or not to an agent which increases the accumulation of said peptide in said cell.

The invention further comprises a kit for treating or preventing metastasis in a subject, said kit comprising at least one peptide consisting essentially of the N2 sequence of the RasGAP protein, a biologically active fragment thereof, or a variant thereof, conjugated or not to an agent which increases the accumulation of said peptide in said cell, optionally with reagents and/or instructions for use.

Also embraced in the scope of the invention is an in vitro method of enhancing the cell adhesion comprising contacting a cell in culture with at least one peptide consisting essentially of the N2 sequence of the RasGAP protein, a biologically active fragment thereof, or a variant thereof, conjugated or not to an agent which increases the accumulation of said peptide in said cell.

Further encompassed is a kit for enhancing the cellular adhesion in vitro, said kit comprising at least one peptide consisting essentially of the N2 sequence of the RasGAP protein, a biologically active fragment thereof, or a variant thereof, conjugated or not to an agent which increases the accumulation of said peptide in said cell, optionally with reagents and/or instructions for use.

The use of a peptide consisting essentially of the N2 sequence of the RasGAP protein, a biologically active fragment thereof, or a variant thereof, conjugated or not to an agent which increases the accumulation of said peptide in a cell for modulating the cell adhesion in vitro is also envisioned.

Further envisioned is the use of a peptide consisting essentially of the N2 sequence of the RasGAP protein, a biologically active fragment thereof, or a variant thereof, conjugated or not to an agent which increases the accumulation of said peptide in a cell as a metastasis inhibitor.

Generally, the kit of the invention comprises a container and a label or package insert on or associated with the container. Suitable containers include, for example, bottles, vials, syringes, etc. The containers may be formed from a variety of materials such as glass or plastic. The container holds a composition which is effective for treating or preventing metastasis and may have a sterile access port (for example the container may be an intravenous solution bag or a vial having a stopper pierceable by a hypodermic injection needle). The label or package insert indicates that the composition is used for treating or preventing metastasis of choice.

Optionally, the kit further comprises a separate pharmaceutical dosage form comprising an anti-cancer agent.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

The foregoing description will be more fully understood with reference to the following Examples. Such Examples, are, however, exemplary of methods of practising the present invention and are not intended to limit the scope of the invention.

### EXAMPLES

### Example 1

### Material & Methods

### Peptides Synthesis

The peptides used in this study were synthetic peptides. The RasGAP-derived peptide TAT-RasGAP₃₁₇₋₃₂₆ (GRKKRRQRRRGGWMWVTNLRTD), the RasGAP₃₁₇₋₃₂₆ (WMWVTNLRTD) peptide and the TAT (HIV-TAT₄₈₋₅₇) peptide (GRKKRRQRRR) were synthesized at the Department of Biochemistry, University of Lausanne, Switzerland using the FMOC technology and purified by HPLC and tested by mass spectrometry (Michod et al., 2004). The R9—RasGAP₃₁₇₋₃₂₆(RRRRRRRRRGGWMWVTNLRTD) and the R9 (RRRRRRRRR) peptides were kind gifts from Dr. Christoph Kündig from MedDiscovery, Switzerland.

### Plasmids

The extension dn3 in the name of a plasmid indicates that the backbone plasmid is the expression vector pcDNA3 (Invitrogen). Plasmid HA-N2.dn3 encodes the haemagglutinin (HA)-tagged human RasGAP amino acid 158-455 sequence (Yang and Widmann, 2001). Plasmid HA-RasGAP₃₁₇₋₃₂₆. dn3 encodes the HA-tagged human RasGAP amino acid 317-326 sequence. It was constructed by cloning the annealed sense (SEQ ID No 18: AATTC GCCCC ATGGGCTACCCGTACGACGTGCCGGACTACGCTTCTTGGATGTGGGTTACAAATTTAAGAACAGAT TAG G) and

anti-sense (SEQ ID No 19: GATCC CTAATCTGTTCTTAAATTTGTAACCCACATCCA AGAAGCGTAGTCCGGCACGTCGTACGGGTAGCCCAT GGGGC G) oligonucleotides into pcDNA3.1(−) (Invitrogen) opened with BamHI and EcoRI.

### Cell Culture

U2OS, HCT116, SAOS and 4T1 cell lines were maintained in Dulbecco's modified essential medium (DMEM+GlutaMAX™, Invitrogen, catalog no: 61965-026) supplemented with 10% foetal bovine serum (FBS, Invitrogen, catalog no: 10270-106) in 3.5 or 10 cm-plates at 37° C. and 5% CO2. HeLa and HEK293T cell lines were maintained in RPMI 1640+GlutaMAX™ (Invitrogen, catalog no: 61870-010) supplemented with 10% FBS. HaCaT cell line were maintained in keratinocyte SFM medium (Invitrogen; catalog no: 17005-042) supplemented with epidermal growth factor 1-53 and extract from bovine pituitary gland (provided with the medium).

### Transfection

2×106 HEK293T cells were cultured overnight in 10 cm-culture plates and transfected with 18 μg of the plasmid of interest and 2 μg of pEGFP-C1 (a green fluorescent protein-encoding plasmid from Clontech) using the calcium-phosphate method (Jordan et al., 1996). Briefly, plasmids were diluted in 450 μl water and mixed with 50 μl CaCl2 2.5M during 30 minutes. After 15 minutes, cells were permeabilized with chloroquine (25 μM final concentration) for 15 minutes. Finally, plasmids were mixed with 500 μl HEP solution (280 mM NaCl, 10 mM KCl, 1.5 mM Na2HPO4, 12 mM D-glucose, 50 mM HEPES) during exactly 1 minute and incubated with the cells during 8 hours at 37° C. and 5% CO2. The medium was then replaced with fresh medium and further incubated for 16-24 hours.

### Adhesion Assay

5×105 cells were grown overnight and then incubated with peptides or transfected (see the figures for the specific conditions used). The treated cells were washed with phosphate buffered saline (PBS) and then incubated with trypsin-EDTA solution (Sigma; catalog no: T3924; 5 mg/ml porcine trypsin, 2 mg/ml EDTA) during 5 minutes. The plates were gently hand-rocked 2 and 4 minute after trypsin addition. Detached cells were removed with a PBS wash and the remaining attached cells were dried, incubated in ethanol during 10 minutes, dried again, and finally stained with GIEMSA (Invitrogen) for 45 minutes. Four and six phase contrast pictures were taken (Zeiss Axioplan, 10× objective) for 3.5 cm-plates and 10 cm-plates, respectively. The number of cells per mm2 was then determined.

### Wound-Healing Experiment

Cells were grown until confluency and a wound in the cell layer was done with a yellow tip. The cells were washed once with PBS to remove debris and then left untreated or incubated 24 hours and 48 hours with 13 μM of TAT or TAT-RasGAP₃₁₇₋₃₂₆ peptides (in DMEM+10% FBS). Pictures were taken just after wounding (0 h) and at the indicated times. The wound widths were measured as described earlier (Bulat et al., 2009).

### Proliferation Assay

30,000 U2OS cells were seeded in 3.5 cm plates and cultured overnight. The cells were either treated with 13 μM TAT, 13 μM TAT-RasGAP317-326 or left untreated. Pictures were taken after 0 h, 24 h and 72 hours and cells were counted. The number of cells was then adjusted per mm2.

### Western Blot Analysis

500,000 U2OS cells were grown overnight in 3.5 cm plates, treated 8 h with 13 μM TAT, TAT-RasGAP317-326 or left untreated, then lysed in 150 μl monoQ-c [70 mM μ-glycerophosphate, 0.5% Triton X-100, 2 mM MgCl2, 1 mM EGTA, 100 μM Na₃VO4, 1 mM dithiothreitol, 20 μg/ml aprotinin, complete EDTA-free Protease Inhibitor Cocktail Tablets (one tablet per 50 ml; Roche Applied Science, Indianapolis, Ind.; catalog no. 1873580)]. The proteins were quantified by a standard Bradford assay and 25 μg were loaded and separated on SDS-PAGE, then blotted onto nitrocellulose membranes (Bio-Ras catalog no. 1620115; BioRad Laboratories, Hercules, Calif.). The membranes were blocked 1 h in 5% BSA in TBS [18 mM HCl, 130 mM NaCl, 20 mM Tris] containing 0.1% Tween (vol/vol) and then incubated overnight in the same solution with the primary antibody c-myc (Cell Signaling; catalog no: 9402) (1:1000). The anti-c-Myc antibody was detected with AlexaFluor 680-conjugated secondary antibodies (Molecular Probes, Eugene, Oreg.; catalog no. A21109) diluted 1:5000 in TBS 0.1% Tween. Membranes were washed after each incubation in TBS 0.1% Tween. Visualization and quantitation were performed using the Odyssey infrared imaging device and software (Licor, Homburg, Germany).

### In Vivo Metastases Model

Nude mice were injected into the mammary fatpads with 100,000 murine mammary cancer 4T1-luc2 cells that constitutively express the firefly luciferase. For the injection, the mouse skin was incised in the pelvic area to reach the fatpads, and the wounds were closed after injection with surgery hooks. The cells were injected in 100 μl Matrigel (BD Biosciences, 20% in PBS). Tumor volumes were quantified as follow: The tumor width and length were measured with a caliper and the tumor volume was calculated according to the formula tumor volume [mm3]=(12*L)*π/6, where 1 is the smallest measure between the width and the length (in mm), and L the longest (in mm) In order to quantify metastases, the mice were injected i.p. with 200 μl D-Luciferin Firefly (15 mg/ml in PBS) in PBS, Biosynth, No. L-8220) and luminescence was measured 10 minutes afterwards with an IVIS Lumina 2 apparatus (Xenogen). Metastases were assessed at the indicated time points in vivo and at the last day (28 days after tumor injection) ex vivo for the following organs: lung, liver and axillary and brachial lymph nodes. For the ex vivo measurements, the mice were injected with luciferin as described above, and sacrificed 10 minutes later to excise organs. The light emitted by the organs was measured in a Luciferin bath (150 μg/ml D-Luciferin Firefly in 2 ml PBS per organ). Luminescence is presented as the number of photons emitted per second (p/s).

### Immuno-Cytochemistry

100,000 U2OS cells were grown on coverslips overnight, and then treated as described in the figures. The cells were then fixed and the nuclei were stained in PBS containing 2% paraformaldehyde (weight/vol) (Acros Organics, catalog no: 30525-89-4) and 10 μg/ml Hoechst 33342 (Molecular Probe) for 15 minutes. The following steps were performed at room temperature in the absence of light. The cells were washed twice in PBS, permeabilized 10 minutes in PBS, 0.2% Triton X-100, washed twice in PBS, neutralized 15 minutes in DMEM, 10% FBS, and washed twice in PBS. The coverslips were then incubated 20 minutes in PBS 1.65 μM Alexa Fluor 488 phalloidin (Invitrogen, A12379). After a rapid wash in PBS, they were incubated with a polyclonal rabbit anti-phospho-FAK (tyrosine 397) primary antibody (1:50 dilution in DMEM, 10% FBS; Cell Signaling, catalog no: 3283) for 1 hour. After a rapid wash in PBS, the coverslips were incubated 1 hour with a donkey CyTM-anti-rabbit secondary antibody (1:500 dilution in DMEM, 10% FBS; Jackson ImmunoResearch, no. 711-165-152). The slides were finally washed twice in PBS and mounted in Vectashield mounting medium (Vector laboratories Inc.). Images were taken with a Zeiss Axioplan 2 imaging microscope.

### Example 2

### Results

### Effect of TAT-RasGAP₃₁₇₋₃₂₆ on Cell Migration

To determine whether the increased adherence induced by TAT-RasGAP₃₁₇₋₃₂₆ would affect the ability of cells to move, a wound-healing experiment was performed with four different cell lines (U2OS, HeLa, HCT116 and HaCaT) (FIG. 2). This experiment revealed that the presence of TAT-RasGAP₃₁₇₋₃₂₆ blocked the ability of the cells to fill wounds. This indicates that TAT-RasGAP₃₁₇₋₃₂₆ has the ability to hamper cell migration. This property, coupled with increased adherence, can be used to inhibit cells from primary tumors to detach and invade other organs. Hence TAT-RasGAP₃₁₇₋₃₂₆ could function as a metastasis inhibitor.

### Effect of TAT-RasGAP317-326 on Cell Proliferation

We next assessed whether the increased adherence and reduced migration induced by TAT-RasGAP₃₁₇₋₃₂₆ affect vital cellular functions. It had already been shown that this peptide does not, by itself, affect cell survival (Michod et al., 2004; Michod et al., 2009). FIG. 3 now shows that the peptide does not modulate cell population growth. Taken together, these results suggest that the effect induced by TAT-RasGAP₃₁₇₋₃₂₆ on adhesion and migration does not adversely affect cells.

TAT-RasGAP₃₁₇₋₃₂₆ is Acting from Inside Cells.

TAT-RasGAP₃₁₇₋₃₂₆ could increase adherence and block migration by acting in the cellular environment or at the surface of cells. Alternatively, these effects could be induced only once the peptide has entered cells. The following evidence favors the second possibility. First, a trypsin inhibitory effect of the peptide could be ruled out because pre-incubating trypsin one hour with TAT-RasGAP₃₁₇₋₃₂₆ did not decrease the ability of trypsin to detach cells (data not shown). Second, the RasGAP₃₁₇₋₃₂₆ sequence without the TAT cell-permeable sequence was unable to increase cell adherence (FIG. 4A). This indicates that when the RasGAP₃₁₇₋₃₂₆ sequence is in the extracellular milieu, it cannot induce cell adherence unless it is hooked to a cell-permeable sequence. Third, transfecting cells with a plasmid encoding the RasGAP₃₁₇₋₃₂₆ sequence also rendered cells more adherent (FIG. 4B), demonstrating that cells can increase their adherence if they synthesize their own RasGAP₃₁₇₋₃₂₆ peptide.

TAT can be Replaced with Other Cell-Permeable Sequences without Altering the Ability of RasGAP₃₁₇₋₃₂₆ to Increase Cell Adherence.

We replaced the TAT sequence with a 9 arginine peptide sequence (R9) in the RasGAP₃₁₇₋₃₂₆ peptide to determine if other cell permeable sequence would allow the RasGAP₃₁₇₋₃₂₆ sequence to be translocated into cells and functions as a cell adherence promoting agent. FIG. 4A shows that this was indeed the case. These results also indicate that the cell-permeable sequences attached to the RasGAP₃₁₇₋₃₂₆ sequence do not contribute to its cellular activity.

### In Vivo TAT-RasGAP₃₁₇₋₃₂₆ Effects on Metastases

We next assessed the potential anti-metastatic ability of RasGAP₃₁₇₋₃₂₆ in the mouse 4T1-luc2 tumor model (Lim et al., 2009). 4T1-luc2 mouse mammary tumor cells constitutively expressing the firefly luciferase gene were orthotopically injected into the mammary fat pad of nude mice. The 4T1 cells develop a local primary tumor and then generate metastases in several organs, including the lungs, liver and lymph nodes (Tao et al., 2008). FIG. 5 shows that RasGAP317-326 did not affect the growth of the primary tumor, as reported in other mouse tumor model (Michod et al., 2009). This is also consistent with the in vitro proliferation assay shown above (FIG. 3).

Twenty-eight days after the injection of 4T1-luc2 cells into the mammary fat pads, the mice were injected intraperitoneally with D-Luciferin, sacrificed, and various organs were subjected to metastases bioluminescence measurements (Table 1). The mice injected with TAT-RasGAP₃₁₇₋₃₂₆ showed a significant decrease in lung metastases (p=0.024 after a Fisher's Exact Test). The peptide had apparently no effect in the formation of metastases in other organs. They suggest however that the TAT-RasGAP₃₁₇₋₃₂₆ peptide could inhibit the metastatic process in some organs (e.g. the lungs).

### Transcription and Translation are not Required for TAT-RasGAP₃₁₇₋₃₂₆ to Increase Cell Adherence

TAT-RasGAP₃₁₇₋₃₂₆-mediated increase in cell adhesion could depend on gene transcription and protein translation. To assess this point, cells were treated with either 1 μg/ml actinomycin D to block transcription or 30 μg/ml cycloheximide to block translation, and then subjected to a trypsin-mediated detachment assay. None of the drugs affected the ability of TAT-RasGAP₃₁₇₋₃₂₆ to increase cell adherence (data not shown). These results indicate that TAT-RasGAP₃₁₇₋₃₂₆ is acting post-translationally.

Metastases Incidence after TAT-RasGAP₃₁₇₋₃₂₆Treatment in Nude Mice Bearing Mammary Gland Cancer.

28 days after cancer cells injection, the same mice than those in FIG. 5 were subjected to ex vivo metastases luminescence measurements. The lung, liver, axillary and brachial lymph node (LN) metastases were quantified by luminescence after luciferine i.p. injection. Organs emitting light were considered as bearing metastases. The asterisk denotes a significant difference against the control treatment (0 mg/kg TAT-RasGAP317-326) (p=0.0238 after an Fisher's Exact Test).

TABLE 1

| | mice with metastases in | | |
|---|---|---|---|
| | the indicated organs | | |
| | vs total number of mice | | |
| Dose (mg/kg) | Lung | Liver | LN |
| 0.000 | 7/7 | 1/7 | 1/7 |
| 0.160 | 2/8* | 3/8 | 3/8 |

### TAT-RasGAP₃₁₇₋₃₂₆ Modulates the Cell's Cytoskeleton

FAK is a key protein in the formation of focal adhesions, which are the sites where integrins and ECM (extracellular matrix) form junctions (Mitra et al., 2005). Integrins are membrane proteins and are involved in several biological processes including predominantly cell migration. They all are heterodimers (α and β subunits) and transduce signals from the external milieu to the interior of the cell (outside-in signaling) but also conversely from the cells to its surface receptors (inside-out signaling). When integrins are bound to their specific ECM, cytoplasmic FAK is recruited and phosphorylated at tyrosine 397, which allows the recruitment of Src and other proteins, leading to the activation of a vast number of downstream signaling events. This, in particular, results in actin remodeling mediated by a Rho-dependent pathway (Guo and Giancotti, 2004).

As TAT-RasGAP₃₁₇₋₃₂₆ affects cell adherence and cell migration, we next determined if it had an effect on focal adhesions and actin fibers that are key cytoskeletal structures in cell adhesion and migration. U2OS cells treated or not with TAT-RasGAP₃₁₇₋₃₂₆ were fixed and stained for phospho-FAK and actin (FIG. 6). Upon TAT-RasGAP₃₁₇₋₃₂₆ treatment, U2OS cells displayed major changes in actin and focal adhesion localization. There was a strong increase in cortical actin stress fibers, while ventral stress fibers almost completely disappeared. Similarly, focal adhesions were mainly found at the cell's periphery. Cells treated with TAT only did not exhibit any difference compared to untreated cells, further confirming that the effect on actin and focal adhesions was specific for the RasGAP₃₁₇₋₃₂₆ sequence.

RasGAP Fragment N2 also Increases Cell Adherence

The RasGAP₃₁₇₋₃₂₆ sequence is found in two of the RasGAP fragments generated by caspase-3: fragment N and fragment N2 (Yang and Widmann, 2001). RasGAP is cleaved in a stepwise manner as caspase activity increases in cells. At low caspase-3 activity, RasGAP is cleaved only once, generating an NH2-terminal fragment, called fragment N, that induces a potent antiapoptotic response (Yang and Widmann, 2001; Yang and Widmann, 2002). At higher caspase activity, fragment N is further processed into two additional fragments, called fragments N1 and N2, that no longer protect cells (Yang and Widmann, 2001; Yang et al., 2005) but that can sensitize tumor cells towards genotoxin-induced apoptosis (Yang et al., 2005). As fragment N2 bears the RasGAP₃₁₇₋₃₂₆ sequence, we determined whether it could also induce cell adherence. As shown in FIG. 4B this was indeed the case. This indicates that the RasGAP₃₁₇₋₃₂₆ sequence can be part of a larger polypeptide and still exerts its cell adhesion promoting activity. As fragment N2 is produced in apoptotic cells, and since the RasGAP₃₁₇₋₃₂₆ sequence modifies the cell's cytoskeleton (see FIG. 6), one can also hypothesizes that fragment N2 plays a role in the changes that apoptotic cells experience at the level of their cellular architecture.

### REFERENCE LIST

Bogenrieder T, Herlyn M. Axis of evil: molecular mechanisms of cancer metastasis. Oncogene 2003 Sep. 29; 22(42):6524-36
Bulat, N., Waeber, G., and Widmann, C. (2009). LDLs stimulate p38 MAPKs and wound healing through SR-BI independently of Ras and PI3 kinase. J. Lipid Res. 50, 81-89.
Guo, W. and Giancotti, F. G. (2004). Integrin signalling during tumour progression. Nat. Rev. Mol. Cell. Biol. 5, 816-826.
Fidler I J. The pathogenesis of cancer metastasis: the ‘seed and soil’ hypothesis revisited. Nat Rev Cancer 2003 June; 3(6):453-8.
Futaki S. et al., “Arginine-rich peptides. An abundant source of membrane-permeable peptides having potential as carriers for intracellular protein delivery” J. Biol. Chem., 276, 5836, 2001
Ioannides C. G. et al., “Inhibition of IL-2 receptor induction and IL-2 production in the human leukemic cell line Jurkat by a novel peptide inhibitor of protein kinase C” Cell Immunol., 131, 242, 1990
Jordan, M., Schallhom, A., and Wurm, F. M. (1996). Transfecting mammalian cells: optimization of critical parameters affecting calcium-phosphate precipitate formation. Nucleic Acids Res. 24, 596-601.
Kole H. K. et al., “A peptide-based protein-tyrosine phosphatase inhibitor specifically enhances insulin receptor function in intact cells” J. Biol. Chem. 271, 14302, 1996
Lim, E., Modi, K. D., and Kim, J. (2009). In vivo bioluminescent imaging of mammary tumors using IVIS spectrum. J. Vis. Exp.
Maniatis et al. 1982, Molecular Cloning, A laboratory Manual, Cold Spring Harbor Laboratory
Michod, D., Annibaldi, A., Schaefer, S., Dapples, C., Rochat, B., and Widmann, C. (2009). Effect of RasGAP N2 fragment-derived peptide on tumor growth in mice. J. Natl. Cancer Inst. 101, 828-832.
Michod, D. and Widmann, C. (2007). TAT-RasGAP₃₁₇₋₃₂₆ requires p53 and PUMA to sensitize tumor cells to genotoxins. Mol. Cancer. Res. 5, 497-507.
Michod, D., Yang, J. Y., Chen, J., Bonny, C., and Widmann, C. (2004). A RasGAP-derived cell permeable peptide potently enhances genotoxin-induced cytotoxicity in tumor cells. Oncogene 23, 8971-8978.
Mitra, S. K., Hanson, D. A., and Schlaepfer, D. D. (2005). Focal adhesion kinase: in command and control of cell motility. Nat. Rev. Mol. Cell. Biol. 6, 56-68.
Pamonsinlapatham, P., Hadj-Slimane, R., Lepelletier, Y., Allain, B., Toccafondi, M., Garbay, C., and Raynaud, F. (2009). P120-Ras GTPase activating protein (RasGAP): a multi-interacting protein in downstream signaling. Biochimie 91, 320-328.
Qian Z. M. et al., “Targeted drug delivery via the transferrin receptor-mediated endocytosis pathway” Pharmacological Reviews, 54, 561, 2002.
Sela M. and Zisman E., “Different roles of D-amino acids in immune phenomena” FASEB J. 11, 449, 1997.
Tao, K., Fang, M., Alroy, J., and Sahagian, G. G. (2008). Imagable 4T1 model for the study of late stage breast cancer. BMC. Cancer 8, 228.
Weigelt B, Peterse J L, van', V. Breast cancer metastasis: markers and models. Nat Rev Cancer 2005 August; 5(8):591-602
Yang, J.-Y., Walicki, J., Michod, D., Dubuis, G., and Widmann, C. (2005). Impaired Akt activity down-modulation, caspase-3 activation, and apoptosis in cells expressing a caspase-resistant mutant of RasGAP at position 157. Mol. Biol. Cell 1 6, 3511-3520.
Yang, J.-Y. and Widmann, C. (2001). Antiapoptotic signaling generated by caspase-induced cleavage of RasGAP. Mol. Cell. Biol. 21, 5346-5358.
Yang, J.-Y. and Widmann, C. (2002). The RasGAP N-terminal fragment generated by caspase cleavage protects cells in a Ras/PI3K/Akt-dependent manner that does not rely on NFκB activation. J. Biol. Chem. 277, 14641-14646.
Zoller M. Tetraspanins: push and pull in suppressing and promoting metastasis. Nat Rev Cancer 2009 January; 9(1):40-55

1. Use of a peptide consisting essentially of the N2 sequence of the RasGAP protein, a biologically active fragment thereof, or a variant thereof, for the preparation of a medicament for the treatment or prevention of metastasis.

2. The use according to claim 1, wherein the biologically active fragment of the N2 sequence of the RasGAP protein comprises the amino acid sequence of the SH3 domain of the N2 sequence, or a variant thereof.

3. The use according to claim 2, wherein the biologically active fragment comprising the amino acid sequence of the SH3 domain of the N2 sequence, or the variant thereof, contains less than or equal to 70 amino acids of the amino acid sequence of the SH3 domain.

4. The use according to claim 1, wherein the biologically active fragment comprising the amino acid sequence of the SH3 domain of the N2 sequence consists in an amino acid sequence selected from the group comprising SEQ ID No. 5, SEQ ID No. 6, SEQ ID No. 7, SEQ ID No. 8, SEQ ID No. 9, aSEQ ID No. 10, SEQ ID No. 11, SEQ ID No. 12, SEQ ID No. 13, SEQ ID No. 14, SEQ ID No. 15 or SEQ ID No. 16.

5. The use according to claim 1, wherein the biologically active fragment comprising the amino acid sequence of the SH3 domain of the N2 sequence comprises the general amino acid sequence WXWVTXXRTX, wherein X represents an amino acid.

6. The use according to claim 1, wherein the biologically active fragment comprising the amino acid sequence of the SH3 domain of the N2 sequence consists in an amino acid sequences encoded by a DNA sequence selected from the group comprising SEQ ID No. 1, SEQ ID No. 2, SEQ ID No. 3 or SEQ ID No. 4.

7. The use according to claim 1, wherein the peptide is conjugated to an agent which increases the accumulation of said peptide in a cell.

8. The use according to claim 7, wherein the agent is a cell membrane permeable carrier.

9. The use according to claim 8, wherein the cell membrane permeable carrier is a peptide.

10. The use according to claim 9, wherein the cell membrane permeable carrier peptide is a positively charged amino acid rich peptide.

11. The use according to claim 10, wherein the positively charged amino acid rich peptide is an arginine rich peptide which is selected from the group comprising the HIV-TAT 48-57 peptide, the FHV-coat 35.49 peptide, the HTLV-II Rex 4_i6 peptide the BMV gag 7.25 peptide and the R9 peptide.

12. The use according to claim 11, wherein the arginine rich peptide is the R9 peptide.

13. The use according to claim 1, wherein the peptide consisting essentially of the N2 sequence of the RasGAP protein, a biologically active fragment thereof, or a variant thereof is either in the L-form or in D-form and/or in a retro-inverso isomer form.

14. The use according to claim 7, wherein the agent which increases the accumulation of the peptide consisting essentially of the N2 sequence of the RasGAP protein, a biologically active fragment thereof, or a variant thereof, is either in the L-form or in D-form and/or in a retro-inverso isomer form.

15. A method of treatment or prevention of metastasis comprising administering to a subject in need thereof, a therapeutically effective amount of i) a peptide consisting essentially of the N2 sequence of the RasGAP protein, a biologically active fragment thereof, or a variant thereof, or ii) a peptide consisting essentially of the N2 sequence of the RasGAP protein, a biologically active fragment thereof, or a variant thereof, conjugated to an agent which increases the accumulation of said peptide in a cell.

16. The method according to claim 15, wherein the biologically active fragment of the N2 sequence of the RasGAP protein comprises the amino acid sequence of the SH3 domain of the N2 sequence, or a variant thereof.

17. The method according to claim 16, wherein the biologically active fragment comprising the amino acid sequence of the SH3 domain of the N2 sequence, or the variant thereof, contains less than or equal to 70 amino acids of the amino acid sequence of the SH3 domain.

18. The method according to claim 15, wherein the biologically active fragment comprising the amino acid sequence of the SH3 domain of the N2 sequence consists in an amino acid sequence selected from the group comprising SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15 or SEQ ID No. 16.

19. The method according to claim 15, wherein the biologically active fragment comprising the amino acid sequence of the SH3 domain of the N2 sequence comprises the general amino acid sequence WXWVTXXRTX, wherein X represents an amino acid.

20. The method according to claim 15, wherein the biologically active fragment comprising the amino acid sequence of the SH3 domain of the N2 sequence consists in an amino acid sequences encoded by a DNA sequence selected from the group comprising SEQ ID No. 1, SEQ ID No. 2, SEQ ID No. 3 or SEQ ID No. 4.

21. The method according to claim 15, wherein the peptide is conjugated to an agent which increases the accumulation of said peptide in a cell.

22. The method according to claim 21, wherein the agent is a cell membrane permeable carrier.

23. The method according to claim 22, wherein the cell membrane permeable carrier is a peptide.

24. The method according to claim 23, wherein the cell membrane permeable carrier peptide is a positively charged amino acid rich peptide.

25. The method according to claim 24, wherein the positively charged amino acid rich peptide is an arginine rich peptide which is selected from the group comprising the HIV-TAT 48-57 peptide, the FHV-coat 35.49 peptide, the HTLV-II Rex 4_i6 peptide the BMV gag [eta]. 25 peptide and the R9 peptide.

26. The method according to claim 25, wherein the arginine rich peptide is the R9 peptide.

27. The method according to claim 15, wherein the peptide consisting essentially of the N2 sequence of the RasGAP protein, a biologically active fragment thereof, or a variant thereof is either in the L-form or in D-form and/or in a retro-inverso isomer form.

28. The method according to claim 15, wherein the agent which increases the accumulation of the peptide consisting essentially of the N2 sequence of the RasGAP protein, a biologically active fragment thereof, or a variant thereof, is either in the L-form or in D-form and/or in a retro-inverso isomer form.

29. An in vivo method of modulating the cell adhesion and cell migration comprising contacting a cell with at least one peptide consisting essentially of the N2 sequence of the RasGAP protein, a biologically active fragment thereof, or a variant thereof, conjugated or not to an agent which increases the accumulation of said peptide in said cell.

30. A kit for treating or preventing metastasis in a subject, said kit comprising at least one peptide consisting essentially of the N2 sequence of the RasGAP protein, a biologically active fragment thereof, or a variant thereof, conjugated or not to an agent which increases the accumulation of said peptide in said cell, optionally with reagents and/or instructions for use.

31. An in vitro method of enhancing the cell adhesion comprising contacting a cell in culture with at least one peptide consisting essentially of the N2 sequence of the RasGAP protein, a biologically active fragment thereof, or a variant thereof, conjugated or not to an agent which increases the accumulation of said peptide in said cell.

32. A kit for enhancing the cellular adhesion in vitro, said kit comprising at least one peptide consisting essentially of the N2 sequence of the RasGAP protein, a biologically active fragment thereof, or a variant thereof, conjugated or not to an agent which increases the accumulation of said peptide in said cell, optionally with reagents and/or instructions for use.

33. Use of a peptide consisting essentially of the N2 sequence of the RasGAP protein, a biologically active fragment thereof, or a variant thereof, conjugated or not to an agent which increases the accumulation of said peptide in a cell for modulating the cell adhesion in vitro.

34. Use of a peptide consisting essentially of the N2 sequence of the RasGAP protein, a biologically active fragment thereof, or a variant thereof, conjugated or not to an agent which increases the accumulation of said peptide in a cell as a metastasis inhibitor.
